(19) United States
(12) Patent Application Publication
(10) Pub. No.: US 2016/0128988 A1
(43) Pub. Date: May 12, 2016

(54) COMBINATIONS FOR THE TREATMENT OF CANCER COMPRISING A MPS-1 KINASE INHIBITOR AND A MITOTIC INHIBITOR

(71) Applicant: BAYER PHARMA AKTIENGESELLSCHAFT, Berlin (DE)

(72) Inventors: Antje Margret WENGNER, Berlin (DE); Gerhard SIEMEISTER, Berlin (DE)

(21) Appl. No.: 14/897,857

(22) PCT Filed: Jun. 6, 2014
(86) PCT No.: PCT/EP2014/061772

§ 371 Date: Dec. 11, 2015

(30) Foreign Application Priority Data
Jun. 11, 2013 (EP) 13171517.9

(51) Int. Cl. A61K 31/437; A61K 33/24; A61K 31/337

(52) U.S. Cl. CPC A61K 31/437; A61K 31/337; A61K 33/24

(57) ABSTRACT

The present invention relates to a combination comprising an Mps-1 kinase inhibitor and a mitotic inhibitor. The present invention also relates to the use of said combination for the treatment of cancer, in particular of pancreatic cancer, glioblastoma, ovarian cancer, non-small cell lung carcinoma, breast cancer and/or gastric cancer.

The present invention relates to a combination comprising an Mps-1 kinase inhibitor and a mitotic inhibitor. The present invention also relates to the use of said combination for the treatment of cancer, in particular of pancreatic cancer, glioblastoma, ovarian cancer, non-small cell lung carcinoma, breast cancer and/or gastric cancer.

### BACKGROUND OF THE INVENTION

Mps-1 (Monopolar Spindle 1) kinase (also known as Tyrosine Threonine Kinase, TTK) is a dual specificity Ser/Thr kinase which plays a key role in the activation of the mitotic checkpoint (also known as spindle checkpoint, spindle assembly checkpoint) thereby ensuring proper chromosome segregation during mitosis [Abrieu A et al., Cell, 2001, 106, 83-93]. Every dividing cell has to ensure equal separation of the replicated chromosomes into the two daughter cells. Upon entry into mitosis, chromosomes are attached at their kinetochores to the microtubules of the spindle apparatus. The mitotic checkpoint is a surveillance mechanism that is active as long as unattached kinetochores are present and prevents mitotic cells from entering anaphase and thereby completing cell division with unattached chromosomes [Suijkerbuijk S J and Kops G J, Biochemica et Biophysica Acta, 2008, 1786, 24-31; Musacchio A and Salmon E D, Nat Rev Mol Cell Biol., 2007, 8, 379-93]. Once all kinetochores are attached in a correct amphitelic, i.e. bipolar, fashion with the mitotic spindle, the checkpoint is satisfied and the cell enters anaphase and proceeds through mitosis. The mitotic checkpoint consists of a complex network of a number of essential proteins, including members of the MAD (mitotic arrest deficient, MAD 1-3) and Bub (Budding uninhibited by benzimidazole, Bub 1-3) families, the motor protein CENP-E, Mps-1 kinase as well as other components, many of these being over-expressed in proliferating cells (e.g. cancer cells) and tissues [Yuan B et al., Clinical Cancer Research, 2006, 12, 405-10]. The essential role of Mps-1 kinase activity in mitotic checkpoint signalling has been shown by shRNA-silencing, chemical genetics as well as chemical inhibitors of Mps-1 kinase [Jelluma N et al., PLos ONE, 2008, 3, e2415; Jones M H et al., Current Biology, 2005, 15, 160-65; Dorer R K et al., Current Biology, 2005, 15, 1070-76; Schmidt M et al., EMBO Reports, 2005, 6, 866-72].

There is ample evidence linking reduced but incomplete mitotic checkpoint function with aneuploidy and tumorigenesis [Weaver B A and Cleveland D W, Cancer Research, 2007, 67, 10103-5; King R W, Biochimica et Biophysica Acta, 2008, 1786, 4-14]. In contrast, complete inhibition of the mitotic checkpoint has been recognised to result in severe chromosome missegregation and induction of apoptosis in tumour cells [Kops G J et al., Nature Reviews Cancer, 2005, 5, 773-85; Schmidt M and Medema R H, Cell Cycle, 2006, 5, 159-63; Schmidt M and Bastians H, Drug Resistance Updates, 2007, 10, 162-81].

Based on these findings, Mps-1 kinase has been considered as one among the most promising drug targets for cancer therapy.

WO2011/064328, WO2011/063907, WO2011/063908, and WO2013/087579A1 relate to [1,2,4]-triazolo-[1,5-a]-pyridines and their use for inhibition of Mps-1 kinase.

Established anti-mitotic drugs such as vinca alkaloids, taxanes or epothilones activate the SAC either by stabilising or destabilising microtubule dynamics resulting in a mitotic arrest. This arrest prevents separation of sister chromatids to form the two daughter cells. Prolonged arrest in mitosis forces a cell either into mitotic exit without cytokinesis or into mitotic catastrophe leading to cell death. In contrast, inhibitors of Mps-1 kinase induce a SAC inactivation that accelerates progression of cells through mitosis resulting in severe chromosomal missegregation and finally in cell death. Silencing of Mps-1 leads to failure of cells to arrest in mitosis in response to anti-mitotic drugs.

Remarkably, combination of microtubule interfering agents and Mps-1 inhibition even increases chromosomal segregation errors and cell death (Abrieu A, Magnaghi-Jaulin L, Kahana J A, Peter M, Castro A, Vigneron S, Lorca T, Cleveland D W, Labbé J C. Mps1 is a kinetochore-associated kinase essential for the vertebrate mitotic checkpoint. Cell 2001; 106: 83-93, Stucke V M, Silljé H H, Arnaud L, Nigg E A. et al. Human Mps-1 kinase is required for the spindle assembly checkpoint but not for centrosome duplication. EMBO J 2002; 21:1723-1732).

Therefore, the combined increase of chromosomal segregation errors induced by combination of anti-mitotics with SAC inhibition constitutes an efficient strategy for selectively eliminating tumor cells.

### SUMMARY OF THE INVENTION

The present invention covers a combination comprising : a compound A of general formula (I):

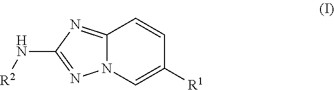

in which:
R¹ represents

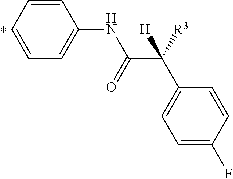

wherein * indicates the point of attachment of said group with the rest of the molecule;
R² represents

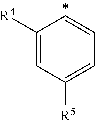

wherein * indicates the point of attachment of said group with the rest of the molecule;
R³ represents a group selected from: methyl-, HO—CH₂—, H₂N—CH₂—, —NH₂;
R⁴ represents a group selected from: methoxy-, F₃C—CH₂—O—;
R⁵ represents a group selected from:

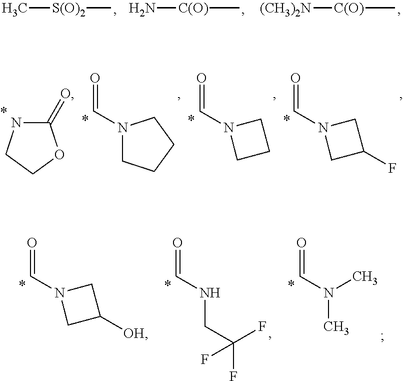

or a hydrate, a solvate, or a salt thereof, or a mixture of same;
and
one or more mitotic inhibitors.

The present invention further relates to the combination as defined supra, for use in the treatment or prophylaxis of cancer, in particular of pancreatic cancer, glioblastoma, ovarian cancer, non-small cell lung carcinoma, breast cancer and/or gastric cancer.

The present invention further relates to the use of the combination as defined supra, for the prophylaxis or treatment of cancer, in particular of pancreatic cancer, glioblastoma, ovarian cancer, non-small cell lung carcinoma, breast cancer and/or gastric cancer.

The present invention further relates to the use of the combination as defined supra, for the preparation of a medicament for the prophylaxis or treatment of cancer, in particular of pancreatic cancer, glioblastoma, ovarian cancer, non-small cell lung carcinoma, breast cancer and/or gastric cancer.

### DETAILED DESCRIPTION OF THE INVENTION

In accordance with a first aspect, the present invention relates to a combination comprising an Mps-1 kinase inhibitor, and one or more mitotic inhibitors.

The Mps-1 kinase inhibitor is selected from the compounds of formula (I):

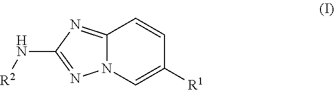

in which:
R¹ represents

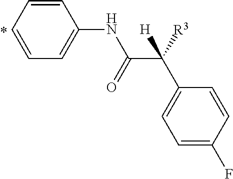

wherein * indicates the point of attachment of said group with the rest of the molecule;
R² represents

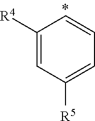

wherein * indicates the point of attachment of said group with the rest of the molecule;
R³ represents a group selected from: methyl-, HO—CH₂—, H₂N—CH₂—, —NH_{2;}
R⁴ represents a group selected from: methoxy-, F3C—CH₂—O—;
and
R⁵ represents a group selected from:

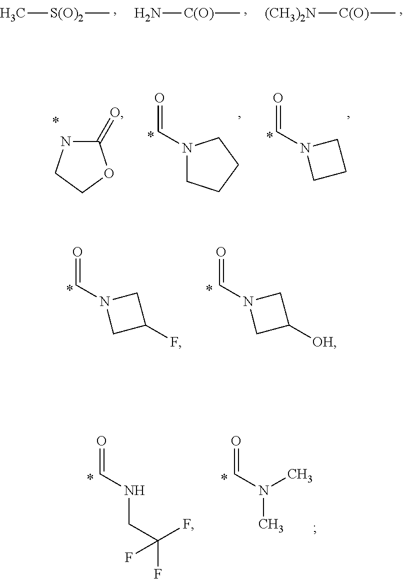

or a hydrate, a solvate, or a salt thereof, or a mixture of same.

In a preferred embodiment, R³ represents a methyl- group.

In another preferred embodiment, R⁴ represents a methoxy- group.

In another preferred embodiment, R⁵ represents a —S(═O)₂CH₃ group.

In another preferred embodiment, the Mps-1 kinase inhibitor is selected from the group consisting of:
(2R)-2-(4-fluorophenyl)-N-[4-(2-{[2-methoxy-4-(methylsulfonyl)phenyl]amino}-[1,2,4]triazolo[1,5-a]pyridin-6-yl)phenyl]propanamide,
(2R)-N-[4-(2-{[2-ethoxy-4-(methylsulfonyl)phenyl]amino}[1,2,4]triazolo[1,5-a]pyridin-6-yl)phenyl]-2-(4-fluorophenyl)propanamide,
(2R)-2-(4-fluorophenyl)-N-[4-(2-{[4-(methylsulfonyl)-2-(2,2,2-trifluoroethoxy)-phenyl]amino}[1,2,4]triazolo[1,5-a]pyridin-6-yl)phenyl]propanamide,
4-{[6-(4-{[(2R)-2-(4-fluorophenyl)propanoyl]amino}phenyl)[1,2,4]triazolo[1,5-a]pyridin-2-yl]amino}-3-methoxy-N-(2,2,2-trifluoroethyl)benzamide,
4-{[6-(4-{[(2R)-2-(4-fluorophenyl)propanoyl]amino}phenyl)[1,2,4]triazolo[1,5-a]pyridin-2-yl]amino}-3-methoxybenzamide,
4-{[6-(4-{[(2R)-2-(4-fluorophenyl)propanoyl]amino}phenyl)[1,2,4]triazolo[1,5-a]pyridin-2-yl]amino}-3-(2,2,2-trifluoroethoxy)benzamide,
(2R)-N-{4-[2-({4-[(3-fluoroazetidin-1-yl)carbonyl]-2-methoxyphenyl}amino)-[1,2,4]triazolo[1,5-a]pyridin-6-]phenyl}-2-(4-fluorophenyl)propanamide,
(2R)-N-[4-(2-{[4-(azetidin-1-ylcarbonyl)-2-methoxyphenyl]amino}[1,2,4]-triazolo[1,5-a]pyridin-6-yl)phenyl]-2-(4-fluorophenyl)propanamide,
(2R)-2-(4-fluorophenyl)-N-[4-(2-{[2-methoxy-4-(2-oxo-1,3-oxazolidin-3-yl)-phenyl]amino}[1,2,4]triazolo[1,5-a]pyridin-6-yl)phenyl]propanamide,
(−)-2-(4-fluorophenyl)-3-hydroxy-N-[4-(2-{[4-(methylsulfonyl)-2-(2,2,2-trifluoroethoxy)phenyl]amino}[1,2,4]triazolo[1,5-a]pyridin-6-yl)phenyl]propanamide,
(2R)-2-amino-2-(4-fluorophenyl)-N-[4-(2-{[2-methoxy-4-(methylsulfonyl)-phenyl]amino}[1,2,4]triazolo[1,5-a]pyridin-6-yl)phenyl]ethanamide,
4-{[6-(4-{[(2R)-2-(4-fluorophenyl)propanoyl]amino}phenyl)[1,2,4]triazolo[1,5-a]pyridin-2-yl]amino}-3-methoxy-N,N-dimethylbenzamide,
(2R)-2-(4-fluorophenyl)-N-[4-(2-{[2-methoxy-4-(pyrrolidin-1-ylcarbonyl)phenyl]amino}[1,2,4]triazolo[1,5-a]pyridin-6-yl)phenyl]propanamide,
(2R)-N-{4-[2-({4-[(3-fluoroazetidin-1-yl)carbonyl]-2-(2,2,2-trifluoroethoxy)phenyl}amino)[1,2,4]triazolo[1,5-a]pyridin-6-yl]phenyl}-2-(4-fluorophenyl)propanamide,
(2R)-2-(4-fluorophenyl)-N-{4-[2-({4-[(3-hydroxyazetidin-1-yl)carbonyl]-2-(2,2,2-trifluoroethoxy)phenyl}amino)[1,2,4]triazolo[1,5-a]pyridin-6-yl]phenyl}propanamide,
(2R)-2-(4-fluorophenyl)-N-[4-(2-{[4-(pyrrolidin-1-ylcarbonyl)-2-(2,2,2-trifluoroethoxy)phenyl]amino}[1,2,4]triazolo[1,5-a]pyridin-6-yl)phenyl]propanamide,
(2S)-2-(4-fluorophenyl)-3-hydroxy-N-[4-(2-{[2-methoxy-4-(methylsulfonyl)phenyl]amino}[1,2,4]triazolo[1,5-a]pyridin-6-yl)phenyl]propanamide,
(2S)-N-{4-[2-({4-[(3-fluoroazetidin-1-yl)carbonyl]-2-(2,2,2-trifluoroethoxy)phenyl}amino)[1,2,4]triazolo[1,5-a]pyridin-6-yl]phenyl}-2-(4-fluorophenyl)-3-hydroxypropanamide,
(2R)-2-amino-2-(4-fluorophenyl)-N-[4-(2-{[4-(methylsulfonyl)-2-(2,2,2-trifluoroethoxy)phenyl]amino}[1,2,4]triazolo[1,5-a]pyridin-6-yl)phenyl]ethanamide,
(2R)-2-amino-2-(4-fluorophenyl)-N-[4-(2-{[2-methoxy-4-(2-oxo-1,3-oxazolidin-3-yl)phenyl]amino}[1,2,4]triazolo[1,5-a]pyridin-6-yl)phenyl]ethanamide,
(2R)-2-amino-N-{4-[2-({4-[(3-fluoroazetidin-1-yl)carbonyl]-2-methoxyphenyl}amino)[1,2,4]triazolo[1,5-a]pyridin-6-yl]phenyl}-2-(4-fluorophenyl)ethanamide,
(2R)-2-amino-N-[4-(2-{[4-(azetidin-1-ylcarbonyl)-2-methoxyphenyl]amino}[1,2,4]triazolo[1,5-a]pyridin-6-yl)phenyl]-2-(4-fluorophenyl)ethanamide,
(2R)-2-amino-2-(4-fluorophenyl)-N-[4-(2-{[2-methoxy-4-(pyrrolidin-1-ylcarbonyl)phenyl]amino}[1,2,4]triazolo[1,5-a]pyridin-6-yl)phenyl]ethanamide,
(2R)-2-amino-N-{4-[2-({4-[(3-fluoroazetidin-1-yl)carbonyl]-2-(2,2,2-trifluoroethoxy)phenyl}amino)[1,2,4]triazolo[1,5-a]pyridin-6-yl]phenyl}-2-(4-fluorophenyl)ethanamide, and
(2R)-2-amino-2-(4-fluorophenyl)-N-[4-(2-{[4-(pyrrolidin-1-ylcarbonyl)-2-(2,2,2-trifluoroethoxy)phenyl]amino}[1,2,4]triazolo[1,5-a]pyridin-6-yl)phenyl]ethanamide,
or a tautomer, an N-oxide, a hydrate, a solvate, or a salt thereof, or a mixture of same.

In another preferred embodiment of the present invention, the Mps-1 kinase inhibitor is (2R)-2-(4-fluorophenyl)-N-[4-(2-{[2-methoxy-4-(methylsulfonyl)-phenyl]amino}[1,2,4]triazolo[1,5-a]pyridin-6-yl)phenyl]propanamide or a tautomer, an N-oxide, a hydrate, a solvate, or a salt thereof, or a mixture of same.

The Mps-1 kinase inhibitor can exist as a hydrate, or as a solvate, wherein the Mps-1 kinase inhibitor contains polar solvents, in particular water, methanol or ethanol for example as structural element of the crystal lattice of the compound. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the Mps-1 kinase inhibitor can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of the Mps-1 inhibitor. For example, see S. M. Berge, et al. “Pharmaceutical Salts,” J. Pharm. Sci. 1977, 66, 1-19.

Further, the Mps-1 kinase inhibitor can exist as an N-oxide, which is defined in that at least one nitrogen of the compound is oxidised. The present invention includes all such possible N-oxides.

Furthermore, the present invention includes all possible crystalline forms, or polymorphs, of the Mps-1 kinase inhibitor, either as single polymorphs, or as a mixture of more than one polymorphs, in any ratio.

In summary, the present invention also relates to useful forms of an Mps-1 kinse inhibitor as disclosed herein. The Mps-1 inhibitor and any useful form of the Mps-1 inhibitor as disclosed herein are also referred to as compound A.

The combination according to the invention further comprises one or more mitotic inhibitors.

The mitotic inhibitor hereinafter is also referred to as compound B.

In a preferred embodiment of the invention, the mitotic inhibitor is a vinca alkaloid, including vinblastine, vincristine, vindesine, vinorelbine, desoxyvincaminol, vincaminol, vinburnine, vincamajine, vineridine, and vinburnine.

In a more preferred embodiment, the mitotic inhibitor is selected from the group consisting of vinblastine, vincristine, vindesine, and vinorelbine.

In an even more preferred embodiment, the mitotic inhibitor is vinorelbine.

In another preferred embodiment of the invention, the mitotic inhibitor is a taxane, including docetaxel, paclitaxel, and their analogues.

Taxanes are known in the art and include, for example, paclitaxel, docetaxel, and the like.

### Paclitaxel:

(2α,4α,5β,7β,10β,13α)-4,10-bis(acetyloxy)-13-{[(2R, 3S)-3-(benzoylamino)-2-hydroxy-3-phenylpropanoyl]oxy}-1,7-dihydroxy-9-oxo-5,20-epoxytax-11-en-2-yl benzoate; commercial names: Taxol, Anzatax, Paxene.

### Docetaxel:

1,7β,10β-trihydroxy-9-oxo-5β,20-epoxytax-11 -ene-2α,4,13α-triyl 4-acetate 2-benzoate 13-{(2R,3S)-3-[(tert-butoxycarbonyl)amino]-2-hydroxy-3-phenylpropanoate}; commercial name: Taxotere.

Taxane based cancer therapy regimens are broadly used in the treatment of ovarian, breast cancer, non-small cell and small cell lung carcinoma, head and neck cancer, esophageal cancer, prostate cancer, bladder cancer and AIDS-related Kaposi's sarcoma. Taxanes, which include paclitaxel, docetaxel and their analogues, are antimicrotubule agents, inhibit microtubule structures within the cell and ultimately cause cell death. Specifically, taxanes such as paclitaxel bind and stabilize microtubules, cause cells to arrest in mitosis and result in cytostatic or cytotoxic responses (E. Chu, et al., ed Cancer Chemotherapy Drug Manual (2010) Jones and Bartlette Publishers.

Other taxanes that become approved by the U.S. Food and Drug Administration (FDA) or foreign counterparts thereof are also preferred for use in the methods and combinations of the present invention. Other taxanes that can be used in the present invention include those described, for example, in 10th NCI-EORTC Symposium on New Drugs in Cancer Therapy, Amsterdam, page 100, Nos. 382 and 383 (Jun. 16-19, 1998); and U.S. Pat. Nos. 4,814,470, 5,721,268, 5,714,513, 5,739,362, 5,728,850, 5,728,725, 5,710,287, 5,637,484, 5,629,433, 5,580,899, 5,549,830, 5,523,219, 5,281,727, 5,939,567, 5,703,117, 5,480,639, 5,250,683, 5,700,669, 5,665,576, 5,618,538, 5,279,953, 5,243,045, 5,654,447, 5,527,702, 5,415,869, 5,279,949, 5,739,016, 5,698,582, 5,478,736, 5,227,400, 5,516,676, 5,489,601, 5,908,759, 5,760,251, 5,578,739, 5,547,981, 5,547,866, 5,344,775, 5,338,872, 5,717,115, 5,620,875, 5,284,865, 5,284,864, 5,254,703, 5,202,448, 5,723,634, 5,654,448, 5,466,834, 5,430,160, 5,407,816, 5,283,253, 5,719,177, 5,670,663, 5,616,330, 5,561,055, 5,449,790, 5,405,972, 5,380,916, 5,912,263, 8,808,113, 5,703,247, 5,618,952, 5,367,086, 5,200,534, 5,763,628, 5,705,508, 5,622,986, 5,476,954, 5,475,120, 5,412,116, 5,916,783, 5,879,929, 5,861,515, 5,795,909, 5,760,252, 5,637,732, 5,614,645, 5,599,820, 5,310,672, RE 34,277, U.S. Pat. Nos. 5,877,205, 5,808,102, 5,766,635, 5,760,219, 5,750,561, 5,637,723, 5,475,011, 5,256,801, 5,900,367, 5,869,680, 5,728,687, 5,565,478, 5,411,984, 5,334,732, 5,919,815, 5,912,264, 5,773,464, 5,670,673, 5,635,531, 5,508,447, 5,919,816, 5,908,835, 5,902,822, 5,880,131, 5,861,302, 5,850,032, 5,824,701, 5,817,867, 5,811,292, 5,763,477, 5,756,776, 5,686,623, 5,646,176, 5,621,121, 5,616,739, 5,602,272, 5,587,489, 5,567,614, 5,498,738, 5,438,072, 5,403,858, 5,356,928, 5,274,137, 5,019,504, 5,917,062, 5,892,063, 5,840,930, 5,840,900, 5,821,263, 5,756,301, 5,750,738, 5,750,562, 5,726,318, 5,714,512, 5,686,298, 5,684,168, 5,681,970, 5,679,807, 5,648,505, 5,641,803, 5,606,083, 5,599,942, 5,420,337, 5,407,674, 5,399,726, 5,322,779, 4,924,011, 5,939,566, 5,939,561, 5,935,955, 5,919,455, 5,854,278, 5,854,178, 5,840,929, 5,840,748, 5,821,363, 5,817,321, 5,814,658, 5,807,888, 5,792,877, 5,780,653, 5,770,745, 5,767,282, 5,739,359, 5,726,346, 5,717,103, 5,710,099, 5,698,712, 5,683,715, 5,677,462, 5,670,653, 5,665,761, 5,654,328, 5,643,575, 5,621,001, 5,608,102, 5,606,068, 5,587,493, 5,580,998, 5,580,997, 5,576,450, 5,574,156, 5,571,917, 5,556,878, 5,550,261, 5,539,103, 5,532,388, 5,470,866, 5,453,520, 5,384,399, 5,364,947, 5,350,866, 5,336,684, 5,296,506, 5,290,957, 5,274,124, 5,264,591, 5,250,722, 5,229,526, 5,175,315, 5,136,060, 5,015,744, 4,924,012, 6,118,011, 6,114,365, 6,107,332, 6,072,060, 6,066,749, 6,066,747, 6,051,724, 6,051,600, 6,048,990, 6,040,330, 6,030,818, 6,028,205, 6,025,516, 6,025,385, 6,018,073, 6,017,935, 6,011,056, 6,005,138, 6,005,138, 6,005,120, 6,002,023, 5,998,656, 5,994,576, 5,981,564, 5,977,386, 5,977,163, 5,965,739, 5,955,489, 5,939,567, 5,939,566, 5,919,815, 5,912,264, 5,912,263, 5,908,835, and 5,902,822, the disclosures of which are incorporated by reference herein in their entirety.

Other compounds that can be used in the invention are those that act through a taxane mechanism. Compounds that act through a taxane mechanism include compounds that have the ability to exert microtubule-stabilizing effects and cytotoxic activity against rapidly proliferating cells, such as tumor cells or other hyperproliferative cellular diseases. Such compounds include, for example, epothilone compounds, such as, for example, epothilone A, B, C, D, E and F, and derivatives thereof. Other compounds that act through a taxane mechanism (e.g., epothilone compounds) that become approved by the FDA or foreign counterparts thereof are also preferred for use in the methods and combinations of the present invention. Epothilone compounds and derivatives thereof are known in the art and are described, for example, in U.S. Pat. Nos. 6,121,029, 6,117,659, 6,096,757, 6,043,372, 5,969,145, and 5,886,026;and WO 97/19086, WO 98/08849, WO 98/22461, WO 98/25929, WO 98/38192, WO 99/01124, WO 99/02514, WO 99/03848, WO 99/07692, WO 99/27890, and WO 99/28324, the disclosures of which are incorporated herein by reference in their entirety.

In a preferred embodiment, the taxane is paclitaxel.

In another preferred embodiment, the taxane is docetaxel.

The combination of the present invention may comprise one or more further pharmaceutical agents. In a preferred embodiment, the combination of the present invention further comprises cisplatin.

Further, the present invention relates to:
a kit comprising:
   a combination of:
component A: one or more Mps-1 kinase inhibitors, as described supra, or a physiologically acceptable salt, solvate, or hydrate thereof;
and
component B: one or more mitotic inhibitors, including docetaxel, paclitaxel, vinblastine, vincristine, vindesine, and vinorelbine;
and, optionally, one or more further pharmaceutical agents C;
in which optionally either or both of said components A and B are in the form of a pharmaceutical formulation which is ready for use to be administered simultaneously, concurrently, separately or sequentially.

The components may be administered independently of one another by the oral, intravenous, topical, local installations, intraperitoneal or nasal route.

The Mps-1 kinase inhibitor is preferably administered orally. The taxane is preferably administered intravenously. The vinca alkaloid is preferably administered intravenously.

Components A and/or B usually are administered in the form of a pharmaceutical composition that is comprised of a pharmaceutically acceptable carrier and a pharmaceutically effective amount of a compound A and/or a compound B of the present invention.

Conventional procedures for preparing such compositions in appropriate dosage forms can be utilized. Ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

The combinations of the present invention can be used for the treatment or prophylaxis of cancer.

In a preferred embodiment, the combinations of the present invention are used for the treatment of pancreatic cancer.

In another preferred embodiment, the combinations of the present invention are used for the treatment of glioblastoma.

In another preferred embodiment, the combinations of the present invention are used for the treatment of non-small cell lung carcinoma.

In another preferred embodiment, the combinations of the present invention are used for the treatment of ovarian cancer.

In another preferred embodiment, the combinations of the present invention are used for the treatment of gastric cancer.

In another preferred embodiment, the combinations of the present invention are used for the treatment of breast cancer.

Combinations of the present invention might be utilized to inhibit, block, reduce, decrease, etc., cell proliferation and/or cell division, and/or produce apoptosis.

The treatment or prohylaxis comprises: administering to a mammal in need thereof, including a human, an amount of a compound A and an amount of compound B of this invention, or a pharmaceutically acceptable salt, isomer, polymorph, hydrate, solvate or ester thereof; etc. which is effective to treat the disorder.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Non-small-cell lung carcinoma (NSCLC) is any type of epithelial lung cancer other than small cell lung carcinoma (SCLC). As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy).

The most common types of NSCLC are squamous cell carcinoma, large cell carcinoma, and adenocarcinoma, but there are several other types that occur less frequently, and all types can occur in unusual histologic variants and as mixed cell-type combinations (“Non-small cell lung cancer treatment—National Cancer Institute”; retrieved 2008 Oct. 19; http://www.cancer.gov/CANCERTOPICS/PDQ/TREATMENT/NON-SMALL-CELL-LUNG/PATIENT).

Lung cancer in never-smokers is almost universally NSCLC, with a sizeable majority being adenocarcinoma.

On relatively rare occasions, malignant lung tumors are found to contain components of both SCLC and NSCLC. In these cases, the tumors should be classified as combined small cell lung carcinoma (c-SCLC), and are (usually) treated like “pure” SCLC.

Breast cancer is a type of cancer originating from breast tissue, most commonly from the inner lining of milk ducts or the lobules that supply the ducts with milk. Cancers originating from ducts are known as ductal carcinomas, while those originating from lobules are known as lobular carcinomas. Breast cancer occurs in humans and other mammals. While the overwhelming majority of human cases occur in women, male breast cancer can also occur. Examples of breast cancer include, but are not limited to invasive ductal carcinoma, invasive lobular carcinoma, ductal carcinoma in situ, and lobular carcinoma in situ.

Ovarian cancer is a cancerous growth arising from the ovary. Most (more than 90%) ovarian cancers are classified as “epithelial” and are believed to arise from the surface (epithelium) of the ovary. However, some evidence suggests that the fallopian tube could also be the source of some ovarian cancers. Since the ovaries and tubes are closely related to each other, it is thought that these fallopian cancer cells can mimic ovarian cancer. Other types may arise from the egg cells (germ cell tumor) or supporting cells.

Gastric cancer, also known as stomach cancer, affects the stomach, which is found in the upper part of the abdomen and just below the ribs. The stomach is part of the body's digestive system. It produces acids and enzymes that break down food before passing it to the small intestine. The cancer can develop in any part of the stomach and spread up towards the esophagus (the tube that connects mouth to the stomach) or down into the small intestine.

Glioblastoma multiforme (GBM), WHO classification name “glioblastoma”, is the most common and most aggressive malignant primary brain tumor in humans, involving glial cells.

Pancreatic cancer is a malignant neoplasm originating from transformed cells arising in tissues forming the pancreas. The most common type of pancreatic cancer is adenocarcinoma (tumors exhibiting glandular architecture on light microscopy) arising within the exocrine component of the pancreas. A minority arise from islet cells, and are classified as neuroendocrine tumors.

### Dose and Administration

Based upon standard laboratory techniques known to evaluate compounds useful for the treatment of hyper-proliferative disorders including cancers, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 20 mg/kg body weight per day.

Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

### Preparation of Compounds of General Formula (I)

Substituted triazolopyridine compounds of general formula (I) can be prepared according to the methods described in WO2013/087579(A1) and WO2014/009219(A1).

### Biological Data

Assays for determining the biological data of compounds of general formula (I) are described in WO2013/087579(A1).

The compounds of general formula (I) are characterized by the following attributes (see WO2013/087579(A1) for more details):
The IC₅₀ determined in an Mps-1 kinase assay with a concentration of 10 μM ATP is lower than or equal to 1 nM.
The IC₅₀ determined in an Mps-1 kinase assay with a concentration of 2 mM ATP is lower than 10 nM. The IC₅₀ of preferred compounds is even lower than 5 nM. The IC₅₀ of more preferred compounds is even lower than 3 nM. The IC₅₀ of most preferred compounds is even lower than 2 nM.
The maximum oral bioavailability (Fₘₐₓ) in rat (determined by means of rat liver microsomes) is higher than 50%. The Fₘₐₓ of preferred compounds is even higher than 70%. The Fₘₐₓ of more preferred compounds is even higher than 80%.
The maximum oral bioavailability (Fₘₐₓ) in dog (determined by means of dog liver microsomes) is higher than 45%. The Fₘₐₓ of preferred compounds is even higher than 52%. The Fₘₐₓ of more preferred compounds is even higher than 70%.
The maximum oral bioavailability (Fₘₐₓ) in human (determined by means of human liver microsomes) is higher than 45%. The Fₘₐₓ of preferred compounds is even higher than 60%. The Fₘₐₓ of more preferred compounds is even higher than 85%.
The IC₅₀ determined in a HeLa cell proliferation assay is lower than 600 nM. The IC₅₀ of preferred compounds is even lower than 400 nM. The IC₅₀ of more preferred compounds is even lower than 200 nM. The IC₅₀ of most preferred compounds is even lower than 100 nM.
   Mode of Action of an Mps-1 Inhibitor as Single-Agent Treatment and in Combination with Paclitaxel

Based on the biological function of Mps-1 kinase in the mitotic SAC induction of micronuclei and/or multinuclearity can be anticipated from an Mps-1 kinase inhibitor. To explain in vivo the mode of action of an Mps-1 kinase inhibitor in single-agent treatment and combination treatment with a taxane, female NMRI (Naval Medical Research Institute) nude mice were implanted with cisplatin-resistant ovarian cancer cells A2780cis.

(2R)-2-(4-fluorophenyl)-N-[4-(2-{[2-methoxy-4-(methylsulfonyl)phenyl]-amino}[1,2,4]triazolo[1,5-a]pyridin-6-yl)phenyl]propanamide (hereinafter referred to as Compound A1) was administered PO (per os, by mouth, oral(ly)) at doses that had induced antitumor efficacy in xenograft models, up to the MTD (maximum tolerated dose) in single-agent treatment (2.5 mg/kg), and in combination treatment (1 mg/kg) in the 2QD (2 times per day) intermittent 2 days on/5 days off dosing schedule. Paclitaxel was administered IV (intravenous(ly)) QW (once per week (meaning the 1 day on/6 days off treatment schedule)) at its MTD (24 mg/kg) in single-agent and combination treatments. Tumor tissue was isolated at different time points after treatment with Compound A1 alone or in combination with paclitaxel. Tumors were embedded in paraffin, sections were prepared and stained with hematoxylin & eosin for histopathological analysis.

Compound A1 single-agent treatment of A2780cis tumors showed induction of a pleomorphic phenotype, including multinuclearity in tumor samples taken 24 hours after the first administration. Paclitaxel treatment induced atypical mitoses 4 and 8 hours after single-agent treatment and increased necrosis and apoptosis was observed in tumor tissues taken 28, 32, and 48 hours after treatment. Combination treatment of Compound A1 with paclitaxel induced atypical mitoses as well as increased pleomorphism and multinucleated tumor cells in A2780cis ovarian tumors (observed in tumor tissue samples taken 32 and 48 hours after the first treatment).

These results showed that Compound A1 inhibited correct distribution of chromosomal material during the cell division cycle, inducing a pleomorphic phenotype in tumor tissue characterized by multinuclearity of tumor cells, whereas paclitaxel induced mitotic arrest, as shown by phenotype of increased atypical mitoses. The observation of different phenotypes after Compound A1 single-agent, paclitaxel single-agent, or Compound A1 with paclitaxel combination treatments indicated the opportunity to use cellular multinuclearity in tumor or surrogate tissues as a pharmacodynamic biomarker to confirm Mps-1 kinase inhibitor activity in Mps-1 kinase inhibitor single-agent or combination treatments with a taxane.

In Vivo Anti-Tumor Efficacy of an Mps-1 Kinase Inhibitor in Combination with Paclitaxel in NCI-H1299 Human NSCLC Model in Nude Mice

The effect of combination treatment of an Mps-1 inhibitor and paclitaxel was studied in the NCI-H1299 taxane intrinsically resistant human lung carcinoma (NSCLC) xenograft model in nude mice.

Compound A1 was applied orally upon sub-optimal doses (18% of MTD) in the optimized twice daily intermittent (2 days on/5 days off) dosing schedule in combination with paclitaxel. Paclitaxel was applied intravenously once per week upon its respective MTD. Substances were formulated in optimal vehicles to achieve solutions. Animal body weight and tumor size were determined two to three times weekly. Treatment for all groups started at a tumor size of 32 mm², at day 10 after tumor cell inoculation. For combination treatment, the Compound A1 and paclitaxel were applied at the same day within a time frame of 4 hours. Animals of control and monotherapy group (Compound A1 only) were treated for a duration of 28 days. Animals of paclitaxel monotherapy and paclitaxel/Mps-1 inhibitor treatment groups were treated for a duration of 37 days. At the end of the study after one final treatment plasma and tumors were sampled for PK analysis and final tumor weight was determined.

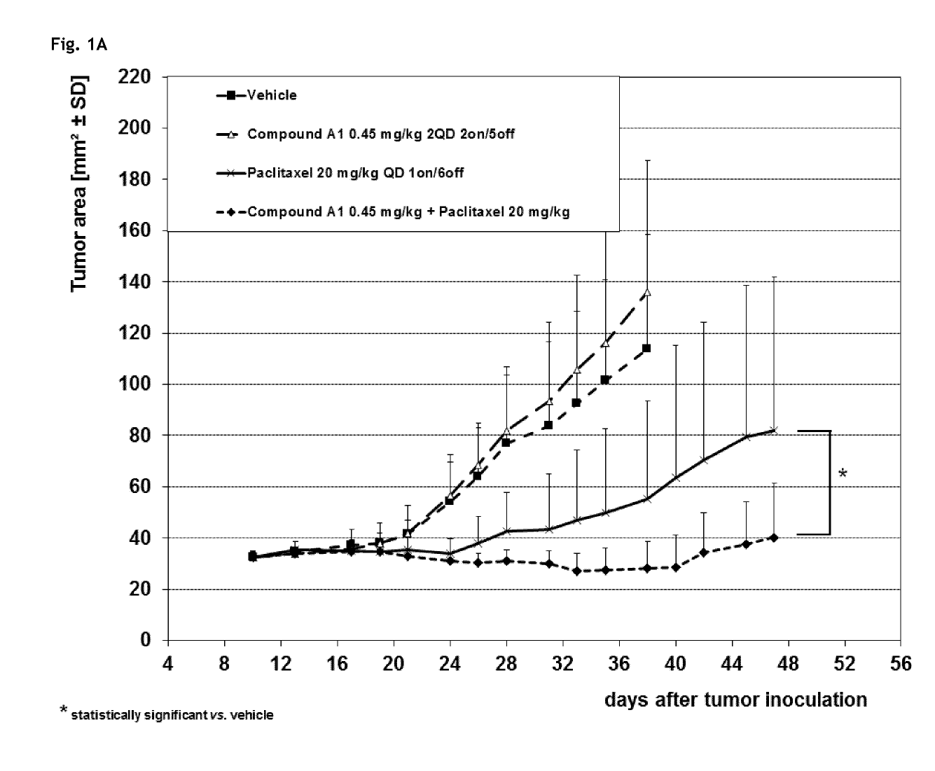

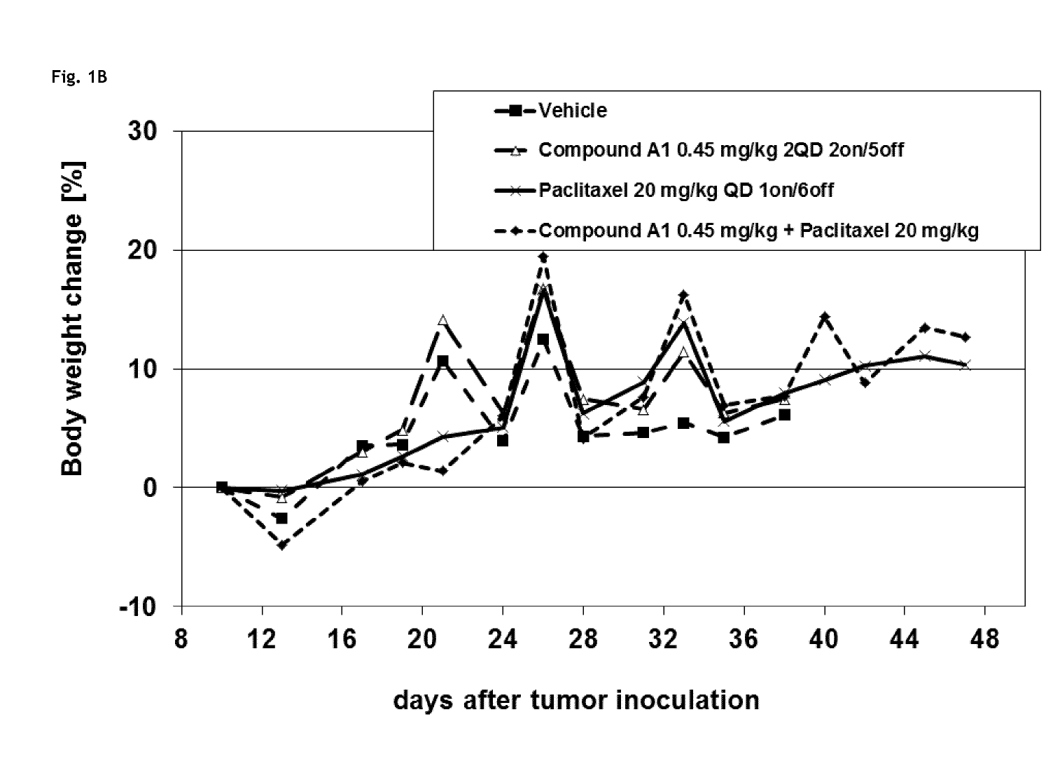

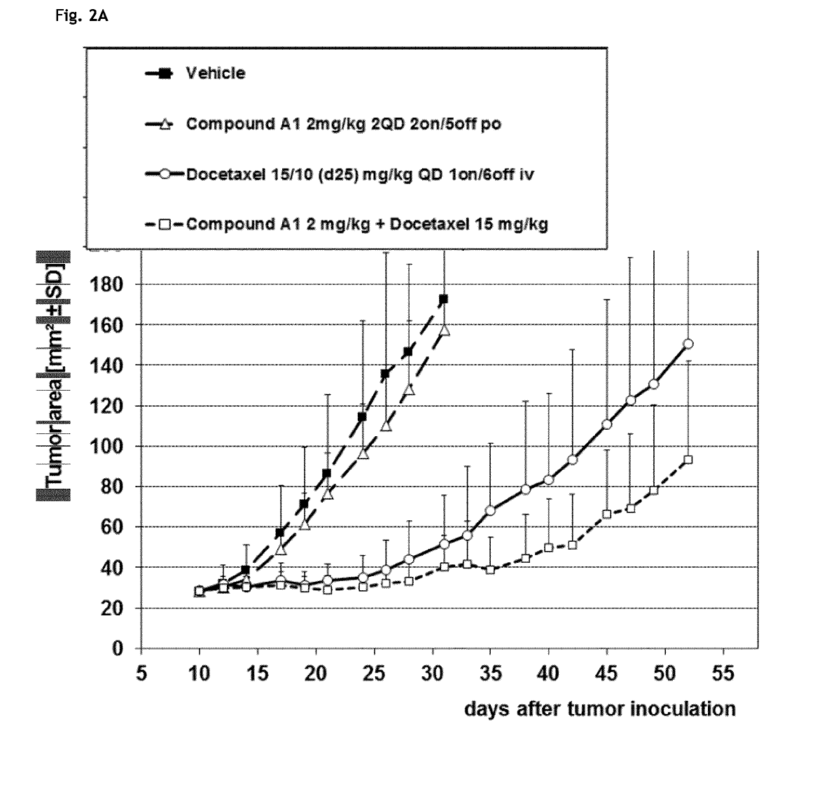

FIGS. 1A and 1B show the response of NCI-H1299 human NSCLC xenograft tumors to treatment with Compound A1 in combination with paclitaxel. NCI-H1299 human NSCLC tumor cells were implanted s.c. into nude mice on day 0. Treatment was started on day 10 when tumors had reached a size of approximately 32 mm². Compound A1 was administered orally (p.o.) upon 0.45 mg/kg twice daily (2QD) for 2 days on/5 days off (2 on/5 off) in monotherapy and in combination with paclitaxel. Paclitaxel was administered intravenously (i.v.) upon 20 mg/kg once daily (QD) once per week (1 on/6 off) in monotherapy and combination therapy. Tumor growth was monitored by determination of the tumor area using calliper measurement two to three times weekly. FIG. 1A: Time course of tumor growth. FIG. 1B: Time course of animal body weight change during the course of the study.

TABLE 1
Mps-1 kinase inhibitor anti-tumor efficacy in combination
with paclitaxel in NCI-H1299 xenoerafts in nude mice.
NCI-H1299 human NSCLC xenograft model

| | | | | Max. | |
|---|---|---|---|---|---|
| | | | | Body | |
| | | | | weight | |
| | Dose and | T/C^{a} | T/C^{a} | loss^{b} | Fatal |
| Compound | Schedule | area | weight | (%) | Tox |
| Vehicle PEG400/ | 10 ml/kg 2QD | 1.00 | 1.00 | −3 | 0/10 |
| Ethanol/ | 2on/5off | | | | |
| Solutol 70:5:25 + | p.o. + | | | | |
| Cremophor 5%/ | 5 ml/kg QD | | | | |
| Ethanol 5%/ | 1on/6off | | | | |
| Saline 90% | i.v. | | | | |
| Compound A1 | 0.45 mg/kg | 1.27 | 1.34 | −1 | 0/10 |
| | 2QD 2on/5off | | | | |
| | p.o. | | | | |
| Paclitaxel | 20 mg/kg QD | 0.28^{#} | — | — | 0/10 |
| | 1on/6off | | | | |
| | i.v. | | | | |
| Compound A1 + | 0.45 mg/kg | −0.05^{#,##} | — | −5 | 0/10 |
| paclitaxel | 2QD 2on/5off | | | | |
| | p.o. + | | | | |
| | 20 mg/kg QD | | | | |
| | 1on/6off | | | | |
| | i.v. | | | | |

^{#}P < 0.05 (compared to vehicle group at day of vehicle group termination)
^{##}P < 0.05 (compared to Paclitaxel group at treatment day 37)
^{a}T/C = Treatment/Control ratio, Calculated from relative mean tumor area at the dosing stop [(tumor area of treatment group at dosing stop) − (tumor area of treatment group at day before first treatment)] or mean final tumor weight.
^{b}Body Weight Loss: the maximum mean body weight loss expressed as a percent of the starting weight of the animal. Weight loss greater than 20% is considered toxic.
PEG 400 = polyethylene glycol having an average molecular weight of 400
Cremophor = polyethoxylated castor oil

Compound A1 showed no effect on tumor growth in monotherapy after 28 treatment days upon dosing with 0.45 mg/kg twice daily (corresponding to 18% of its monotherapy MTD) for 2 days on/5 days off p.o. (FIG. 1A, Table 1).

Both, paclitaxel monotherapy upon its MTD (20 mg/kg) applied for 1 day on/6 days off i.v. and paclitaxel (20 mg/kg, 1 on/6 off, i.v.) combination therapy with Compound A1, dosed upon 18% of its MTD with 0.45 mg/kg twice daily p.o. for 2 days on/5 days off, achieved statistically significant reduction of tumor size compared to vehicle treated control group after 28 treatment days, showing relative T/Cₐᵣₑₐ of 0.28 for paclitaxel monotherapy and relative T/Cₐᵣₑₐ of −0.05 for paclitaxel/Mps-1 kinase inhibitor combination therapy. Paclitaxel monotherapy and paclitaxel/Mps-1 kinase inhibitor combination groups were treated for another 9 days. After 37 treatment days statistically significant improvement of paclitaxel monotherapy efficacy was achieved in the paclitaxel/Mps-1 kinase inhibitor combination treatment group. Although progressive disease was observed in both paclitaxel monotherapy and paclitaxel/Mps-1 kinase inhibitor combination treatment groups, signs of disease stabilization could be detected upon combination treatment, achieving overall a clear tumor growth delay compared to paclitaxel monotherapy in NCI-H1299 human lung tumors (FIG. 1A, Table 1).

Mps-1 kinase inhibitor (Compound A1) as well as paclitaxel monotherapy and combination treatments were all well tolerated without critical body weight loss or toxicities (FIG. 1B, Table 1).

In summary, this study demonstrates cooperativity of Mps-1 kinase inhibitor and paclitaxel in the paclitaxel intrinsically resistant human NCI-H1299 lung cancer (NSCLC) model, achieving significant tumor growth delay compared to paclitaxel monotherapy.

In Vivo Anti-Tumor Efficacy of an Mps-1 Kinase Inhibitor in Combination with Docetaxel in NCI-H1299 Human NSCLC Model in Nude Mice

The effect of combination treatment of an Mps-1 kinase inhibitor (Compound A1) with docetaxel, another SAC activating, microtubule-destabilizing, anti-mitotic agent, used as standard of care in NSCLC patients, was studied in the NCI-H1299 taxane intrinsically resistant human lung carcinoma (NSCLC) xenograft model in nude mice.

Compound A1 was applied orally upon sub-optimal doses (80% of MTD) in the optimized twice daily intermittent (2 days on/5 days off) dosing schedule in combination with docetaxel. Docetaxel was applied intravenously once per week upon its respective MTD. Substances were formulated in optimal vehicles to achieve solutions. Animal body weight and tumor size were determined two times weekly. Treatment for all groups started at a tumor size of 28 mm², at day 10 after tumor cell inoculation. For combination treatment Compound A1and docetaxel were applied at the same day within a time frame of 4 hours. Animals of control and Mps-1 kinase inhibitor monotherapy group were treated for a duration of 20 days. Animals of docetaxel monotherapy and docetaxel/Mps-1 kinase inhibitor combination treatment groups were treated for a duration of 42 days. At the end of the study after one final treatment plasma and tumors were sampled for PK analysis and final tumor weight was determined.

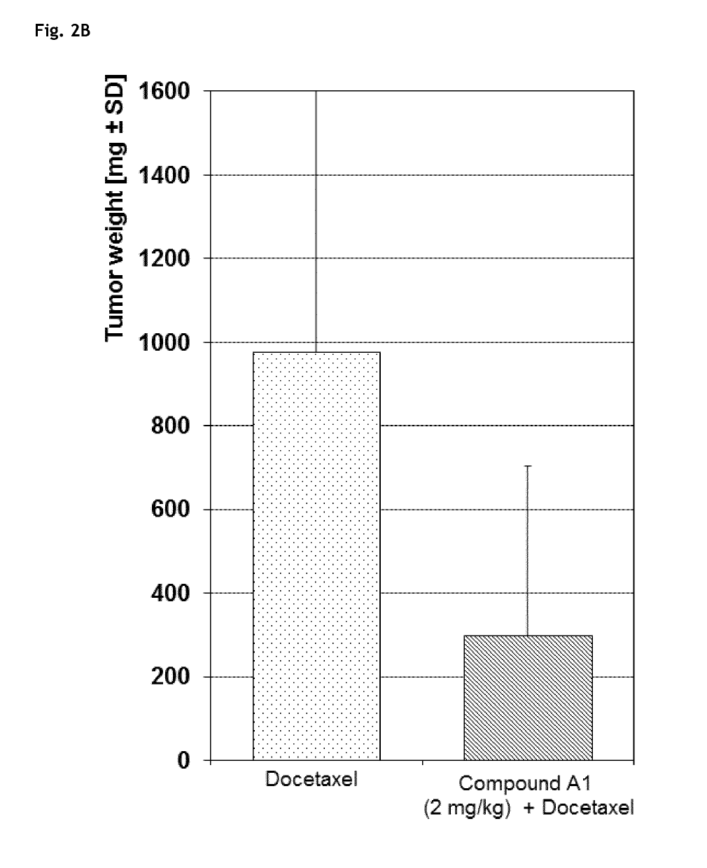

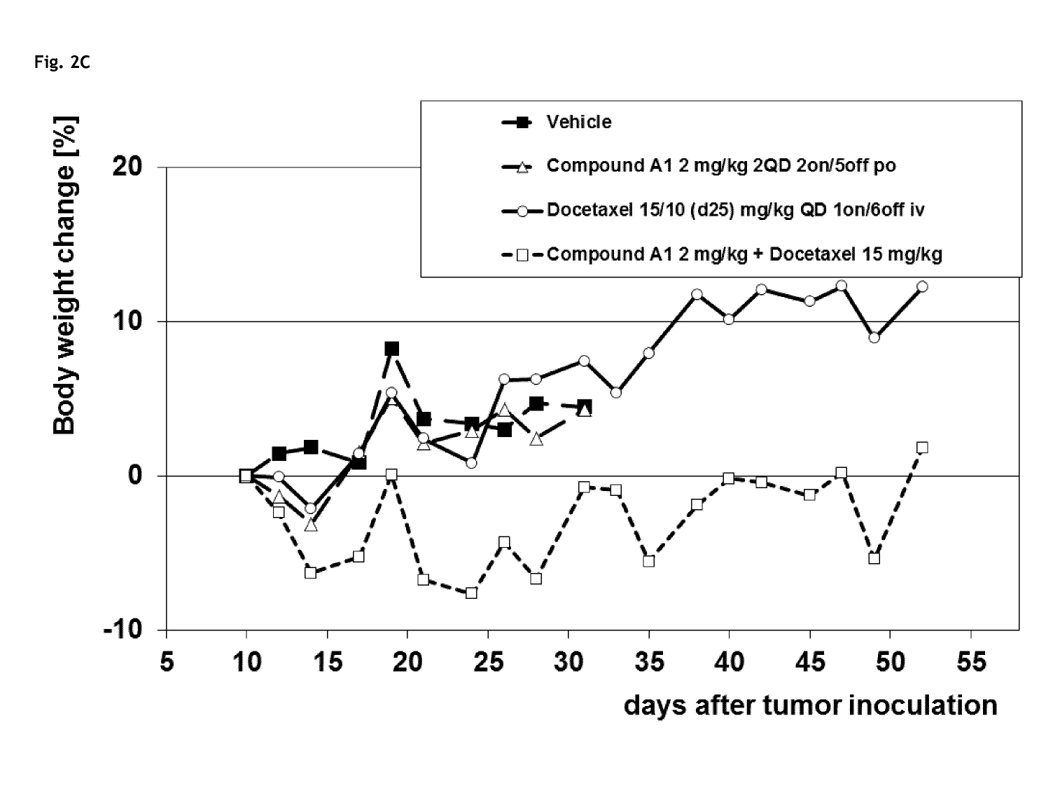

FIGS. 2A, 2B, and 2C show the response of NCI-H1299 human NSCLC xenograft tumors to treatment with Compound A1 in combination with docetaxel. NCI-H1299 human NSCLC tumor cells were implanted s.c. into nude mice on day 0. Treatment was started on day 10 when tumors had reached a size of approximately 28 mm². Compound A1 was administered orally (p.o.) upon 2.0 mg/kg twice daily (2QD) for 2 days on/5 days off (2 on/5 off) in monotherapy and in combination with docetaxel. Docetaxel was administered intravenously (i.v.) upon 15 mg/kg once daily (QD) once per week (1 on/6 off) in monotherapy and combination therapy. Tumor growth was monitored by determination of the tumor area using calliper measurement two to three times weekly. FIG. 2A: Time course of tumor growth. FIG. 2B: Weight of tumors in docetaxel/Mps-1 kinase inhibitor combination group at the end of the study at day 52 after tumor cell inoculation. FIG. 2C: Time course of animal body weight change during the course of the study.

TABLE 2
Mps-1 kinase inhibitor anti-tumor efficacy in combination
with docetaxel in NCI-H1299 xenografts in nude mice.
NCI-H1299 human NSCLC xenograft model

| | | | | Max. | |
|---|---|---|---|---|---|
| | | | | Body | |
| | | | | weight | |
| | Dose and | T/C^{a} | T/C^{a} | loss^{b} | Fatal |
| Compound | Schedule | area | weight | (%) | Tox |
| Vehicle PEG400/ | 10 ml/kg 2QD | 1.00 | 1.00 | — | 0/8 |
| Ethanol/ | 2on/5off | | | | |
| Solutol 70:5:25 + | p.o. + | | | | |
| Saline | 5 ml/kg QD | | | | |
| | 1on/6off | | | | |
| | i.v. | | | | |
| Compound A1 | 2 mg/kg 2QD | 0.89 | 1.01 | −3 | 0/8 |
| | 2on/5off | | | | |
| | p.o. | | | | |
| Docetaxel | 15 mg/kg QD | 0.16^{#} | — | −2 | 0/8 |
| | 1on/6off | | | | |
| | i.v. | | | | |
| Compound A1 + | 2 mg/kg 2QD | 0.08^{#} | — ^{##} | −8 | 0/8 |
| Docetaxel | 2on/5off | | | | |
| | p.o. + | | | | |
| | 15 mg/kg QD | | | | |
| | 1on/6off | | | | |
| | i.v. | | | | |

^{#}P < 0.05 (compared to vehicle group at day of vehicle group termination)
^{##} P < 0.05 (compared to docetaxel group at treatment day 42)
^{a}T/C = Treatment/Control ratio, Calculated from relative mean tumor area at the dosing stop [(tumor area of treatment group at dosing stop) − (tumor area of treatment group at day before first treatment)] or mean final tumor weight.
^{b}Body Weight Loss: the maximum mean body weight loss expressed as a percent of the starting weight of the animal. Weight loss greater than 20% is considered toxic.

Compound A1 achieved weak efficacy in monotherapy after 20 treatment days upon dosing at 80% of its monotherapy MTD (2 mg/kg) twice daily for 2 days on/5 days off p.o. (FIG. 2A, Table 2).

Both, docetaxel monotherapy upon its MTD (15 mg/kg) applied for 1 day on/6 days off i.v. and docetaxel (15 mg/kg, 1 on/6 off, i.v.) combination therapy with Compound A1, dosed upon 80% of its MTD with 2 mg/kg twice daily p.o. for 2 days on/5 days off, achieved statistically significant reduction of tumor size compared to vehicle treated control group after 20 treatment days, showing relative T/Cₐᵣₑₐ of 0.16 for docetaxel monotherapy and relative T/Cₐᵣₑₐ of 0.08 for docetaxel/Mps-1 kinase inhibitor combination therapy. Docetaxel monotherapy and docetaxel/Mps-1 kinase inhibitor combination groups were treated for another 21 days. After 42 treatment days statistically significant improvement of docetaxel monotherapy efficacy was achieved in the docetaxel/Mps-1 kinase inhibitor combination treatment group. Although progressive disease was observed in both docetaxel monotherapy and docetaxel/Mps-1 kinase inhibitor combination treatment groups, overall a clear tumor growth delay compared to docetaxel monotherapy was achieved in combination treatment with Compound A1 in NCI-H1299 human lung tumors (FIG. 2A, FIG. 2B, Table 2).

Mps-1 kinase inhibitor as well as docetaxel monotherapy treatments were well tolerated, without critical body weight loss or toxicities. Tolerability of docetaxel/Mps-1 kinase inhibitor combination was acceptable without toxicities. Here, maximum body weight of −1 to −8% was observed after treatment cycles (FIG. 2C, Table 2).

In summary, this study demonstrates that also combination of an Mps-1 kinase inhibitor with docetaxel, another SAC activating, microtubule-destabilizing, anti-mitotic agent, achieves cooperativity. In addition, as well as in combination with paclitaxel, significant improvement of tumor growth delay compared to docetaxel monotherapy could be achieved in taxane intrinsically resistant human lung cancer (NSCLC).

In Vivo Anti-Tumor Efficacy of an Mps-1 Kinase Inhibitor in Combination with Paclitaxel in MDA-MB 231 Human Triple-Negative Breast Cancer Model in Nude Mice

The effect of combination treatment of an Mps-1 kinase inhibitor (Compound A1) and paclitaxel was studied in the MDA-MB 231 human triple-negative (no expression of Her2/neu, progesterone receptor, estrogen receptor) xenograft model in nude mice.

Compound A1 was applied orally upon sub-optimal doses (40% of MTD) in the optimized twice daily intermittent (2 days on/5 days off) dosing schedule in combination with paclitaxel. Paclitaxel was applied intravenously once per week upon its respective MTD. Substances were formulated in optimal vehicles to achieve solutions. Animal body weight and tumor size were determined three times weekly. Treatment for all groups started at a tumor size of 27 mm², at day 24 after tumor cell inoculation. For combination treatment, Compound A1 and paclitaxel were applied at the same day within a time frame of 4 hours. Animals of control and monotherapy group (Compound A1 only) were treated for a duration of 28 days. Animals of paclitaxel monotherapy and paclitaxel/Mps-1 kinase inhibitor treatment groups were treated for a duration of 50 days. At the end of the study after one final treatment plasma and tumors were sampled for PK analysis and final tumor weight was determined.

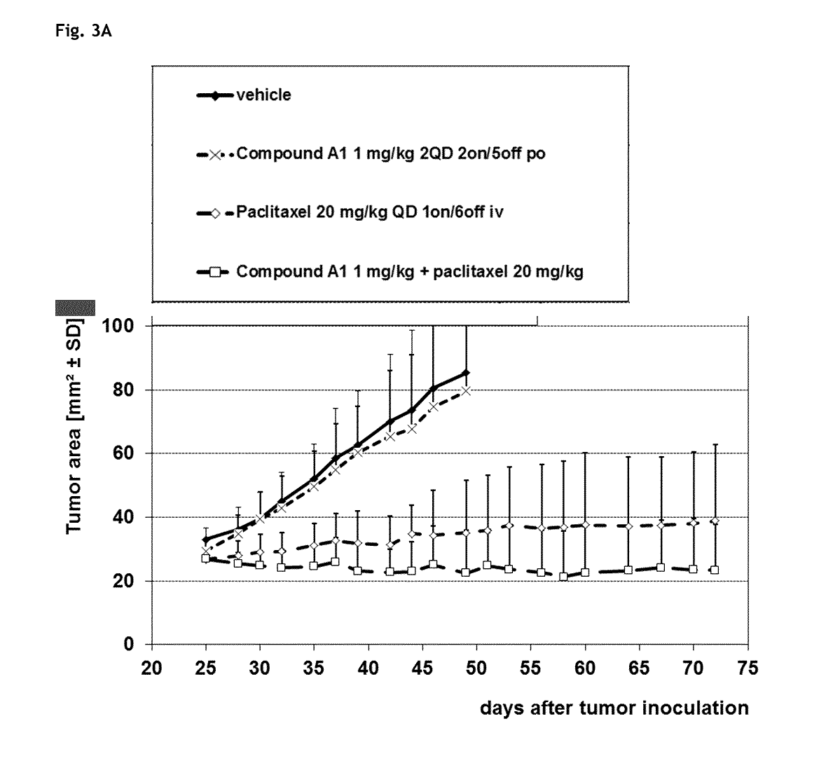

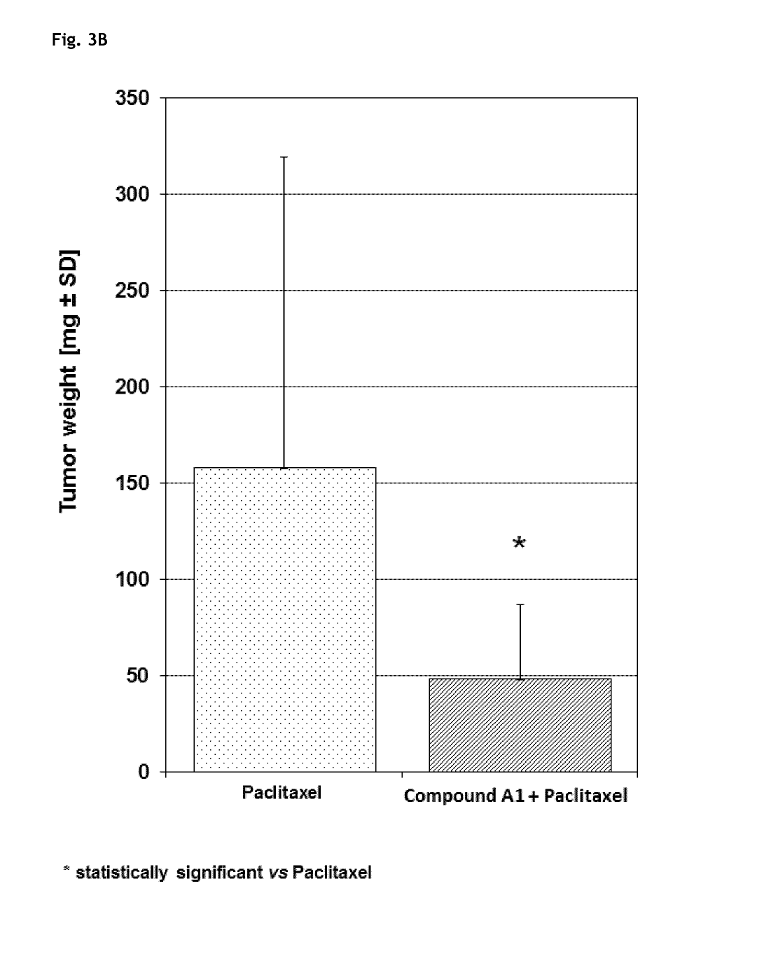

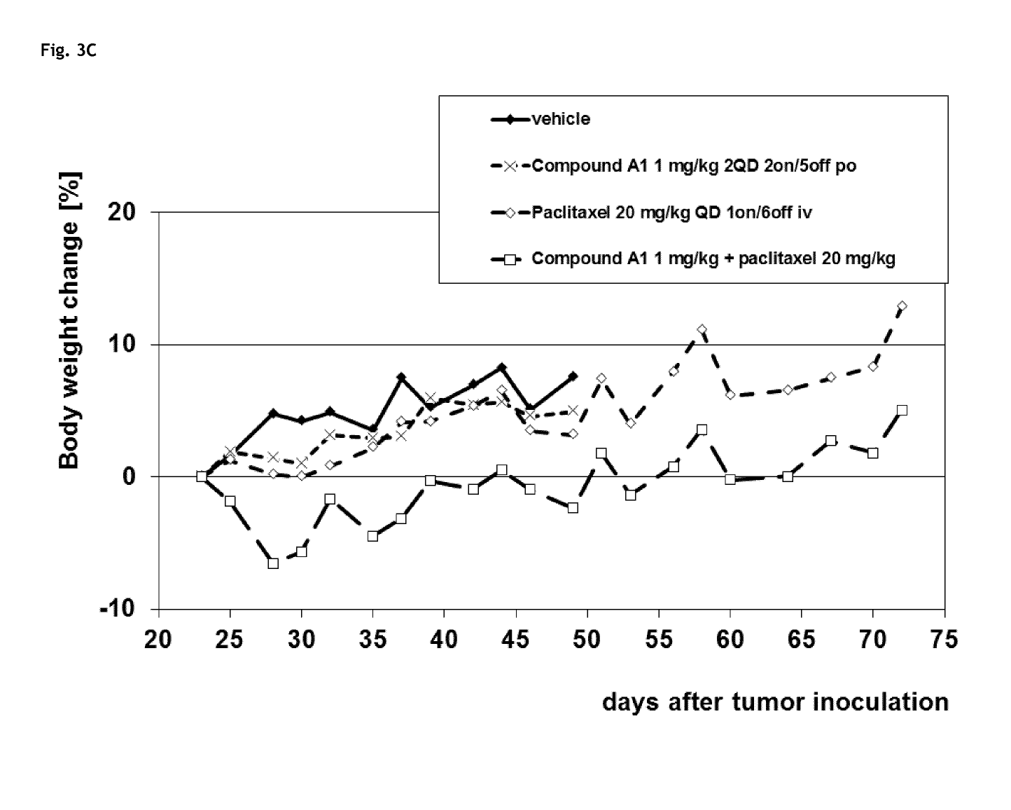

FIGS. 3A, 3B, and 3C show the response of MDA-MB 231 human triple-negative breast cancer xenograft tumors to treatment with Compound A1 in combination with paclitaxel. MDA-MB 231 human breast cancer cells were implanted s.c. into nude mice on day 0. Treatment was started on day 24 when tumors had reached a size of approximately 27 mm². Compound A1 was administered orally (p.o.) upon 1 mg/kg twice daily (2QD) for 2 days on/5 days off (2 on/5 off) in monotherapy and in combination with paclitaxel. Paclitaxel was administered intravenously (i.v.) upon 20 mg/kg once daily (QD) once per week (1 on/6 off) in monotherapy and combination therapy. Tumor growth was monitored by determination of the tumor area using calliper measurement three times weekly. FIG. 3A: Time course of tumor growth. FIG. 3B: Weight of tumors in paclitaxel/Mps-1 kinase inhibitor combination group at the end of the study at day 73 after tumor cell inoculation. FIG. 3C: Time course of animal body weight change during the course of the study.

TABLE 3
Mps-1 kinase inhibitor anti-tumor efficacy in combination
with paclitaxel in MDA-MB 231 xenografts in nude mice.
MDA-MB 231 human triple-negative breast cancer xenograft model

| | | | | Max. | |
|---|---|---|---|---|---|
| | | | | Body | |
| | | | | weight | |
| | Dose and | T/C^{a} | T/C^{a} | loss^{b} | Fatal |
| Compound | Schedule | area | weight | (%) | Tox |
| Vehicle PEG400/ | 10 ml/kg 2QD | 1.00 | 1.00 | — | 0/10 |
| Ethanol/ | 2on/5off | | | | |
| Solutol 70:5:25 + | p.o. + | | | | |
| Cremophor 5%/ | 5 ml/kg QD | | | | |
| Ethanol 5%/ | 1on/6off | | | | |
| Saline 90% | i.v. | | | | |
| Compound A1 | 1 mg/kg 2QD | 0.91 | 0.92 | — | 0/10 |
| | 2on/5off | | | | |
| | p.o. | | | | |
| Paclitaxel | 20 mg/kg QD | 0.14^{#} | — | — | 0/10 |
| | 1on/6off | | | | |
| | i.v. | | | | |
| Compound A1 + | 1 mg/kg 2QD | −0.07^{#,##} | — | −6 | 0/10 |
| Paclitaxel | 2on/5off | | | | |
| | p.o. + | | | | |
| | 20 mg/kg QD | | | | |
| | 1on/6off | | | | |
| | i.v. | | | | |

^{#}P < 0.05 (compared to vehicle group at day of vehicle group termination)
^{##}P < 0.05 (compared to Paclitaxel group at treatment day 73)
^{a}T/C = Treatment/Control ratio, Calculated from relative mean tumor area at the dosing stop [(tumor area of treatment group at dosing stop) − (tumor area of treatment group at day before first treatment)] or mean final tumor weight.
^{b}Body Weight Loss: the maximum mean body weight loss expressed as a percent of the starting weight of the animal. Weight loss greater than 20% is considered toxic.

Compound A1 showed no effect on tumor growth in monotherapy after 28 treatment days upon dosing at 40% of its monotherapy MTD (1 mg/kg) twice daily for 2 days on/5 days off p.o. (FIG. 3A, Table 3).

Both, paclitaxel monotherapy upon its MTD (20 mg/kg) applied for 1 day on/6 days off i.v. and paclitaxel (20 mg/kg, 1 on/6 off, i.v.) combination therapy with Compound A1, dosed upon 40% of its MTD with 1 mg/kg twice daily p.o. for 2 days on/5 days off, achieved statistically significant reduction of tumor size compared to vehicle treated control group after 28 treatment days, showing relative T/Cₐᵣₑₐ of 0.14 for paclitaxel monotherapy and relative T/Cₐᵣₑₐ of −0.07 for paclitaxel/Mps-1 kinase inhibitor combination therapy. Paclitaxel monotherapy and paclitaxel/Mps-1 kinase inhibitor combination groups were treated for another 22 days. After 50 treatment days statistically significant improvement of paclitaxel monotherapy efficacy was achieved in the paclitaxel/Mps-1 kinase inhibitor combination treatment group. Clear disease stabilization was observed upon combination treatment with paclitaxel and Mps-1 kinase inhibitor, achieving a clear improvement of tumor growth control compared to paclitaxel monotherapy in the MDA-MB 231 human triple-negative breast cancer model (FIG. 3A, FIG. 3B, Table 3).

Mps-1 kinase inhibitor (Compound A1) as well as paclitaxel monotherapy and combination treatments were all well tolerated without critical body weight loss or toxicities (FIG. 3C, Table 3).

In summary, this study demonstrates cooperativity of Mps-1 kinase inhibitor and paclitaxel in the human triple-negative breast cancer model MDA-MB 231, showing significant improvement of paclitaxel monotherapy and achieving clear disease stabilization.

Combination of an Mps-1 Inhibitor with Paclitaxel in the Primary Human NSCLC Model LU387

The effect of Compound A1 and paclitaxel compared with a vehicle was evaluated in the subcutaneous primary human NSCLC xenograft model LU387 on nude mice.

The mean tumor size of the vehicle-treated mice reached 1516 mm³ on day 42 post treatment. Treatment with Compound A1 at 1 mg/kg and 2.5 mg/kg (PO, 2QD, 2 days on/5 days off over 6 weeks) produced mean tumor size of 1788 and 1137 mm³, respectively, at the same time. The low dose did not produce any antitumor activity. The high dose produced some tumor inhibition with T/Cᵥₒₗᵤₘₑ of 0.73, which was not significant (P=0.552). Treatment with paclitaxel at 20 mg/kg (IV; QW) for 6 weeks produced mean tumor size of 1494 mm³ (P=1.000, compared with the vehicle-treated group). The combination treatment with Compound A1 and paclitaxel produced mean tumor size of 741 mm³ on day 42 (P=0.054, compared with the vehicle-treated group), more effective than any single-agent treatment. The tumor weight result is consistent with the tumor volume data. Based on body weight changes, the treatments were well tolerated by the animals.

However, Compound A1 as a single agent at doses of 1 mg/kg and 2.5 mg/kg showed no significant antitumor activity against the primary human NSCLC xenograft model LU387. In contrast, the Compound A1 with paclitaxel combination treatment showed more additive efficacy in this study.

Combination of an Mps-1 Kinase Inhibitor with Paclitaxel in IGR-OV1 Human Ovarian Cancer Model

The effect of combination treatment of Compound A1 with the SAC activating, microtubule-stabilizing, antimitotic agent paclitaxel was studied in the IGR-OV1 human ovarian carcinoma model in nude mice. Compound A1 was administered PO up to the respective MTD in single-agent treatment and at 80% of the single-agent MTD in combination in the 2QD for 2 days on/5 days off dosing schedule. Paclitaxel was administered IV QW at 50% of its respective MTD. Treatment for all groups started at a tumor size of 27 mm², on day 5 after tumor cell inoculation. For combination treatment, Compound A1 and paclitaxel were administered on the same day within a time of 4 hours. Animals of control and Compound A1 single-agent treatment groups were treated for 38 days. Animals of paclitaxel single-agent treatment and Compound A1 with paclitaxel combination treatment groups were treated for 77 days.

Compound A1 achieved moderate single-agent efficacy and statistically significant improvement of tumor growth inhibition compared with the vehicle-treated control group after 38 treatment days on MTD dosing with 2.5 mg/kg PO 2QD for 2 days on/5 days off, achieving T/C_{weight} of 0.52 and relative T/Cₐᵣₑₐ of 0.57. Paclitaxel single-agent treatment at 50% of its MTD (12 mg/kg IV QW) and paclitaxel (same dose and schedule) combination treatment with Compound A1, dosed at 80% of its single-agent MTD with 2 mg/kg PO 2QD for 2 days on/5 days off, achieved statistically significant reduction of tumor size compared with the vehicle-treated control group after 38 treatment days, with relative T/Cₐᵣₑₐ of 0.34 for paclitaxel single-agent treatment and relative T/Cₐᵣₑₐ of 0.07 for Compound A1 with paclitaxel combination treatment. A statistically significant improvement compared to the paclitaxel single-agent treatment was achieved in the Compound A1 with paclitaxel combination treatment group, increasing from treatment day 38 to treatment day 59, when the paclitaxel single-agent treatment group showed clear tumor progression, whereas the Compound A1 with paclitaxel combination treatment group induced disease stabilization in 50% and partial regression in 10% of IGR-OV1 tumors. From treatment day 59 onwards, large (80 mm²) progressively growing tumors from the paclitaxel single-agent treatment group were treated with Compound A1 (in addition to paclitaxel) at 2 mg/kg PO 2QD for 2 days on/5 days off for another 2 treatment cycles (tumor growth observation for another 18 days). This combination treatment of paclitaxel-insensitive tumors induced clear tumor growth stagnation and disease stabilization, indicating that growth of large paclitaxel pre-treated, refractory tumors was inhibited by addition of Compound A1 to paclitaxel single-agent treatment. Compound A1 as well as paclitaxel single-agent treatments and combination treatment were all well tolerated without critical body weight loss or toxicities.

In summary, this study showed clear cooperativity of an Mps-1 kinase inhibitor and paclitaxel in the ovarian carcinoma model IGR-OV1. Furthermore, the study demonstrated, that progressive taxane-refractory tumor growth can be inhibited by addition of an Mps-1 kinase inhibitor to paclitaxel pre-treated large tumors.

Combination of an Mps-1 Kinase Inhibitor with Paclitaxel in the A2780cis Human Ovarian Cancer Model

The effect of the combination treatment of Compound A1 with paclitaxel was studied in a human ovarian carcinoma model in nude mice, namely in the adaptive cisplatin-resistant and paclitaxel intrinsically-resistant A2780cis xenograft model. Compound A1 was administered PO at a dose of 40% of the single-agent MTD in combination treatment in the intermittent 2QD 2 days on/5 days off dosing schedule. Paclitaxel was administered IV QW at its respective MTD. Treatment for all groups started at a tumor size of 26 mm², on day 6 after tumor cell inoculation. For combination treatment, Compound A1 and paclitaxel were administered on the same day within a time of 4 hours. Animals of the control and Compound A1 single-agent treatment groups were treated for 14 days. Animals of the paclitaxel single-agent treatment and Compound A1 with paclitaxel combination treatment groups were treated for 36 days.

Compound A1 achieved weak single-agent efficacy after 14 treatment days at 40% of the single-agent MTD dosing with 1 mg/kg PO 2QD for 2 days on/5 days off, achieving T/C_{weight} of 0.87 and relative T/Cₐᵣₑₐ of 0.89. Both, paclitaxel single-agent treatment at its MTD (24 mg/kg IV QW) and paclitaxel (24 mg/kg IV QW) combination treatment with Compound A1 at 1 mg/kg PO 2QD for 2 days on/5 days off, achieved statistically significant reductions of tumor size compared with the vehicle-treated control group after 14 treatment days, showing relative T/Cₐᵣₑₐ of 0.09 for paclitaxel single-agent treatment and relative T/Cₐᵣₑₐ of 0.03 for Compound A1 with paclitaxel combination treatment. Paclitaxel single-agent treatment and Compound A1 with paclitaxel combination treatment groups were treated for another 22 days. After 36 treatment days, statistically significant improvement of the paclitaxel single-agent efficacy was achieved in the Compound A1 with paclitaxel combination treatment group. Although progressive disease was observed in both paclitaxel single-agent and Compound A1 with paclitaxel combination treatment groups, a clear tumor growth delay on combination treatment compared to paclitaxel single-agent treatment was observed in A2780cis ovarian tumors. Compound A1 as well as paclitaxel single-agent and combination treatments were all well tolerated without critical body weight loss or toxicities.

In summary, this study demonstrated cooperativity of an Mps-1 kinase inhibitor and paclitaxel in the paclitaxel intrinsically-resistant ovarian carcinoma model A2780cis, achieving significant tumor growth delay compared to paclitaxel single-agent treatment.

Combination of an Mps-1 Kinase Inhibitor with Cisplatin and Paclitaxel (Triple Combination) in the A2780 Human Ovarian Cancer Model

The effect of the combination treatment of Compound A1 with cisplatin and paclitaxel was studied in the A2780 human ovarian carcinoma model in nude mice. Cisplatin with paclitaxel combination therapy is currently used as SoC in ovarian and lung cancer patients. The A2780 model had previously been demonstrated to be insensitive to cisplatin with paclitaxel combination treatment administered at the combination-treatment MTD. The aim of this study was to investigate, whether the triple combination treatment of Compound A1 with cisplatin and paclitaxel can overcome insensitivity/resistance. Compound A1 was administered PO up to its single-agent MTD and at a lower dose of 40% thereof in the combination treatment. The 2QD intermittent 2 days on/5 days off dosing schedule was used. Cisplatin and paclitaxel were administered in combination at their respective MTD, cisplatin intraperitoneally (IP) every second week (Q2W) and paclitaxel IV QW. Treatment for all groups started at a tumor size of 27 mm², on day 4 after tumor cell inoculation. For combination treatment, Compound A1, paclitaxel, and cisplatin were administered on the same day within a time of 4 hours. Animals of the control and Compound A1 single-agent treatment groups were treated for 8 days. Animals of cisplatin with paclitaxel and triple combination (Compound A1 with cisplatin and paclitaxel) treatment groups were treated for 17 days.

Compound A1 achieved weak single-agent efficacy after 8 treatment days at 40% of its MTD (1 mg/kg) and at its MTD (2.5 mg/kg) 2QD for 2 days on/5 days off PO. Cisplatin with paclitaxel combination treatment at their MTD (cisplatin at 4.5 mg/kg Q2W IP and paclitaxel at 18 mg/kg IV QW) as well as the triple combination treatment group with cisplatin and paclitaxel at their MTD and Compound A1 at 40% of its single-agent MTD (1 mg/kg PO 2QD for 2 days on/5 days off) achieved statistically significant reduction of tumor size compared with the vehicle-treated control group after 8 treatment days, with relative T/Cₐᵣₑₐ of 0.14 for cisplatin with paclitaxel and relative T/Cₐᵣₑₐ of 0.03 for Compound A1 with cisplatin and paclitaxel triple combination treatment. After 17 treatment days, statistically significant improvement over cisplatin with paclitaxel efficacy was observed in the triple combination treatment group. Although tumor progression was observed in both cisplatin with paclitaxel and Compound A1 with cisplatin and paclitaxel triple combination treatment groups, the triple combination showed strong tumor growth delay compared to cisplatin with paclitaxel combination treatment, with clear signs of disease stabilization in A2780 human ovarian tumors. Cisplatin with paclitaxel as well as Compound A1 with cisplatin and paclitaxel combination treatments were well tolerated without critical body weight loss or toxicities. Transient body weight loss (maximum: −3 to −7%) was observed from treatment day 4 to 13 in both combination groups, indicating that this body weight loss was induced by cisplatin with paclitaxel combination treatment.

In summary, this study demonstrated clear cooperativity of an Mps-1 kinase inhibitor in triple combination with cisplatin and paclitaxel in the cisplatin/paclitaxel intrinsically-insensitive human ovarian carcinoma model A2780, achieving significant tumor growth delay compared to the cisplatin with paclitaxel SoC combination treatment.

Combination of an Mps-1 Kinase Inhibitor with Paclitaxel in the MKN1 Human Gastric Cancer Model

The effect of Compound A1 with paclitaxel combination treatment was studied in the taxane-sensitive MKN1 human gastric carcinoma model in nude mice. Compound A1 was administered PO in the 2QD intermittent (2 days on/5 days off) dosing schedule up to the respective MTD in single-agent treatment and at a dose of 40% of the single-agent MTD in combination. Paclitaxel was administered IV QW at its respective MTD. Treatment for all groups started at a tumor size of 27 mm², on day 7 after tumor cell inoculation. For combination treatment, Compound A1 and paclitaxel were administered on the same day within a time of 4 hours. Animals of the control and Compound A1 single-agent treatment groups were treated for 40 days. Animals of the paclitaxel single-agent and Compound A1 with paclitaxel combination treatment groups were treated for 78 days. Vehicle-treated control and Compound A1 single-agent treatment groups had to be terminated before reaching maximum tumor area (at 80 to 90 mm²) due to MKN1 tumor-associated cachexia, inducing critical body weight loss and toxicity.

Compound A1 showed no effect on tumor growth inhibition in single-agent treatment after 40 treatment days at a dose of 40% of the single-agent MTD (1 mg/kg) or at the MTD (2.5 mg/kg) administered 2QD for 2 days on/5 days off PO. Both, paclitaxel single-agent treatment at its MTD (24 mg/kg IV QW) and paclitaxel (24 mg/kg IV QW) combination treatment with Compound A1 at 1 mg/kg PO 2QD for 2 days on/5 days off, achieved clear and statistically significant reduction of tumor growth compared with the vehicle-treated control group after 40 treatment days, showing relative T/Cₐᵣₑₐ of 0.05 for paclitaxel single-agent treatment and relative T/Cₐᵣₑₐ of −0.12 for Compound A1 with paclitaxel combination treatment. Paclitaxel single-agent and Compound A1 with paclitaxel combination treatment groups were treated for another 38 days. At the end of the study, after 78 treatment days, the paclitaxel single-agent treatment group achieved overall disease stabilization, with 80% tumor growth delay and 20% partial regression. Compound A1 with paclitaxel combination treatment induced 75% tumor regression, including 1 complete regression, in the MKN1 tumor model. This combination treatment showed also statistically significant improvement of paclitaxel single-agent efficacy. Paclitaxel single-agent and combination treatments were overall well tolerated without critical body weight loss. Fatal toxicity occurred in 1 animal of the paclitaxel single-agent treatment group on Treatment Day 57 as well as in 2 animals of the Compound A1 with paclitaxel combination treatment group after the first treatment cycle.

In summary, this study demonstrated cooperativity of an Mps-1 kinase inhibitor and paclitaxel in the paclitaxel-sensitive gastric carcinoma model MKN1. Remarkably, combination treatment at doses below the single-agent MTD of Compound A1 with paclitaxel at its MTD significantly improved overall disease stabilization, inducing tumor regressions as compared to paclitaxel single-agent treatment.

Combination of an Mps-1 Kinase Inhibitor with Vincristine in the in the MiaPaCa2 Human Pancreatic Tumor Model

The aim of the study was the evaluation of efficacy and tolerability of Mps-1 kinase inhibitor Compound A1 in combination with Paclitaxel in the MiaPaCa2 human pancreatic tumor model xenografted onto nude mice.

MiaPaCa2 cells obtained from cell culture were implanted s.c. into the inguinal region of female nude mice. Treatment was started when the tumors were 30-40 mm² in size. Tumor area was determined by caliper measurements twice weekly. Treatment groups were:
1) vehicle, PEG400/Ethanol/Solutol (70:5:25), bid 2 on/5 off p.o.
2) Compound A1, 0.45 mg/kg bid 2 on/5 off p.o.
3) Compound A1, 0.6 mg/kg bid 2 on/5 off p.o.
4) Paclitaxel, 24.0 mg/kg i.v. od 1 on/6 off
5) Compound A1, 0.45 mg/kg bid 2 on/5 off p.o.+Paclitaxel, 24.0 mg/kg i.v. od 1 on/6off
6) Compound A1, 0.6 mg/kg bid 2 on/5 off p.o.+Paclitaxel, 24.0 mg/kg i.v. od 1 on/6 off

In this study, significant improvement of tumor growth inhibition by combination of Paclitaxel dosed at its MTD (24 mg/kg od 1 day on/6 days off) with suboptimal dose of Mps-1 inhibitor Compound A1 (0.6 mg/kg bid 2 days on/5 days off) could be demonstrated compared to Paclitaxel monotherapy in the MiaPaCa2 pancreatic tumor model. Tumor growth delay (TGD) of combination treatment to Paclitaxel monotherapy at 100 mm² is about 21 days.

No critical body weight loss occurred. Acute toxicity was observed in the 2 combination treatment groups (tox 2/8, tox 1/8).

No differences in plasma concentrations of Paclitaxel could be detected in combination treatment with Paclitaxel and Compound A1 compared to Paclitaxel treatment alone.

In summary, significant improvement of tumor growth inhibition by combination of Paclitaxel at MTD with low dose of Compound A1 at good tolerability compared to Paclitaxel monotherapy in the Taxane semi-sensitive pancreatic tumor model MiaPaCa2 could be demonstrated.

Combination of an Mps-1 Kinase Inhibitor with Vincristine in the in the Human Glioblastoma Model U87 MG

The dose dependent tumor-inhibiting effects of Compound A1 alone or in combination with vincristine were investigated in the human glioblastoma model U87 MG, xenografted in nude mice.

The study was designed to determine the response of this glioblastoma model to the treatment with the investigational Compound A1 and vincristine, both alone at a fixed dose, and vincristine in combination with two different doses, both lower than that of Compound A1 used in the monotherapy schedule. The size of the glioblastoma was used as read out parameter for response.

The cell culture derived human xenograft U87 MG was initiated by transplantation of the tumor cells into the left hemisphere of the mouse brain. Treatment was done in three cycles between day 3 and day 19. Mice were sacrificed at day 24, the brain isolated and shock frozen in 2 methyl-butane. Tumor size were determined as measure for tumor growth inhibition from cryo-slizes after staining.

The treatment with all drugs were tolerated without a significant body weight loss. Only mice of gr. A and B had a visible body weight loss at the end of the experiment, caused by the tumor. Several mice from several groups including the control group had severe diarrhea, indicating gastro-intestinal toxicity, in some cases with lethal results, indicating an intolerance against the vehicle. In the lethal cases, the isolation of tumor was complicated or impossible. The tumor model caused several sudden death during, but mostly at the end of the experiment, when the general condition of the mouse got unpredictable worse overnight.

The cerebral tumor grew in 16 mice not only inside the brain cranium but also extracranial. Both areas were measured and summed up for analysis. Results are summarized in Table 4.

Compound A1 as single drug had no inhibitory effect in the schedule and at the dose of 2.5 mg/kg used for treatment of U87 MG glioma growth. Only mice treated with vincristine alone or in combination with Compound A1 showed an inhibition of tumor growth between 38.6% (Gr. C; vincristine alone) and 64.0% (Gr. D, combination of high dose Compound A1 and vincristine). The inhibition of tumor growth in Gr. D was significantly different to control group (p<0.05) based on the size of the intracerebral tumor area. Much better is the result, if inner- and outercerebral tumor area together is compared to the other groups. The inhibition was significantly different to control group (p<0.05) and to Gr. B and C with p<0.01 (data from Table 4).

In summary, the combination of Compound A1 with vincristine resulted in a significant inhibition of tumor growth in the human U87-MG mouse xenograft, which was better than the treatment with the single drugs. The treatment was accompanied with severe gastrointestinal toxicity which was most likely caused by intolerance against the vehicle.

TABLE 4
Study summary, tumor growth inhibition (T/C), and tolerability (BWC)

| | | | | | | | | Brain | | | T/C |
|---|---|---|---|---|---|---|---|---|---|---|---|
| | | | | Dose | | | BWC1 | tumor | | | (inside |
| | Mice | | | (mg/kg/ | Treatment | | [%] | inside | outside | total | T) |
| Group | n(n)² | Substance | Route | inj) | Schedule | Days | (at day) | [mm²] | [mm²] | [mm²] | [%] |
| A | 8(4) | Vehicle | p.o. | — | BIDx2x3 | 3, 4, 10, | −1.1 (d24) | 17.40 +/− 4.08 | | 17.40 +/− 4.08 | 100.0 |
| | | | | | | 11, 18, 19 | | | | | |
| B | 8(7) | Compound | p.o. | 2.5 | BIDx2x3 | 3, 4, 10, | −7.7 (d24) | 15.89 +/− 9.93 | 5.50 +/− 4.09 | 20.64 +/− 12.14 | 91.3 |
| | | A1 | | | | 11, 18, 19 | | | | | |
| C | 8(7) | Vincristine | i.p. | 0.75 | 1xqWx3 | 3, 10, 18 | — | 10.69 +/− 6.71 | 4.50 +/− 1.66 | 13.26 +/− 7.25 | 61.4 |
| D | 8(6) | Compound | p.o. | 1.25 | BIDx2x3 | 3, 4, 10, | −1.1 (d6) | 6.26 +/− 3.97* | 4.00 | 6.93 +/− 5.29** | 36.0 |
| | | A1 | | | | 11, 18, 19 | | | | | |
| | | Vincristine | i.p. | 0.75 | 1xqWx3 | 3, 10, 18 | | | | | |
| E | 8(8) | Compound | p.o. | 0.8 | BIDx2x3 | 3, 4, 10, | — | 10.49 +/− 8.13 | 5.67 +/− 2.89 | 12.61 +/− 7.72 | 60.3 |
| | | A1 | | | | 11, 18, 19 | | | | | |
| | | Vincristine | i.p. | 0.75 | 1xqWx3 | 3, 10, 18 | | | | | |

1Nadir of body weight
²Number of mice at start (number of mice used for tumor size quantification)
Vehicle: PEG400/Ethanol/Solutol
*significantly different to vehicle treated group; p < 0.05;
**significantly different to control (Gr. A), p < 0.05, and to Gr. B, C p < 0.1
BID: twice a day
qW: once per week

1. A combination comprising:
a compound A of general formula (I): 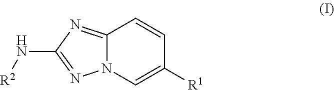
in which:
R¹ represents 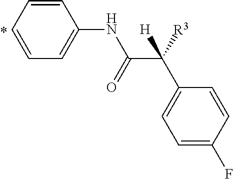
wherein * indicates the point of attachment of said group with the rest of the molecule;
R² represents 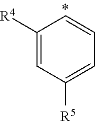
wherein * indicates the point of attachment of said group with the rest of the molecule;
R³ represents a group selected from: methyl-, HO—CH₂—, H₂N—CH₂—, and —NH₂;
R⁴ represents a group selected from: methoxy-, and F₃C—CH₂—O—;
R⁵ represents a group selected from: 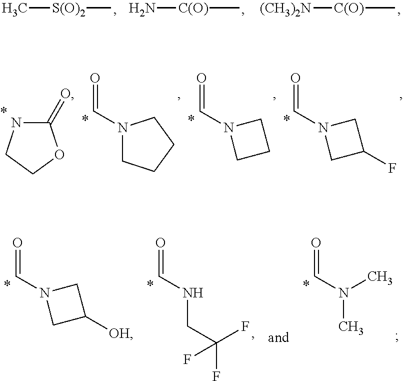
or a hydrate, a solvate, or a salt thereof, or a mixture of same;
and
one or more mitotic inhibitors.

2. The combination according to claim 1, wherein the mitotic inhibitor is a vinca alkaloid.

3. The combination according to claim 1, wherein the mitotic inhibitor is a taxane.

4. The combination according to claim 1, wherein the mitotic inhibitor is selected from docetaxel and paclitaxel.

5. The combination according to claim 1, wherein
R³ represents a methyl- group.
R⁴ represents a methoxy- group; and
R⁵ represents a 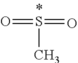
group;
wherein * indicates the point of attachment of said group with the rest of the molecule.

6. The combination according to claim 1, wherein compound A is selected from:
(2R)-2-(4-fluorophenyl)-N-[4-(2-{[2-methoxy-4-(methylsulfonyl)phenyl]amino}-[1,2,4]triazolo[1,5-a]pyridin-6-yl)phenyl]propanamide,
(2R)-N-[4-(2-{[2-ethoxy-4-(methylsulfonyl)phenyl]amino}[1,2,4]triazolo[1,5-a]pyridin-6-yl)phenyl]-2-(4-fluorophenyl)propanamide,
(2R)-2-(4-fluorophenyl)-N-[4-(2-{[4-(methylsulfonyl)-2-(2,2,2-trifluoroethoxy)-phenyl]amino}[1,2,4]triazolo[1,5-a]pyridin-6-yl)phenyl]propanamide,
4-{[6-(4-{[(2R)-2-(4-fluorophenyl)propanoyl]amino}phenyl)[1,2,4]triazolo[1,5-a]pyridin-2-yl]amino}-3-methoxy-N-(2,2,2-trifluoroethyl)benzamide,
4-{[6-(4-{[(2R)-2-(4-fluorophenyl)propanoyl]amino}phenyl)[1,2,4]triazolo[1,5-a]-pyridin-2-yl]amino}-3-methoxybenzamide,
4-{[6-(4-{[(2R)-2-(4-fluorophenyl)propanoyl]amino}phenyl)[1,2,4]triazolo[1,5-a]-pyridin-2-yl]amino}-3-(2,2,2-trifluoroethoxy)benzamide,
(2R)-N-{4-[2-({4-[(3-fluoroazetidin-1-yl)carbonyl]-2-methoxyphenyl}amino)-[1,2,4]triazolo[1,5-a]pyridin-6-yl]phenyl}-2-(4-fluorophenyl)propanamide,
(2R)-N-[4-(2-{[4-(azetidin-1-ylcarbonyl)-2-methoxyphenyl]amino}[1,2,4]triazolo[1,5-a]pyridin-6-yl)phenyl]-2-(4-fluorophenyl)propanamide,
(2R)-2-(4-fluorophenyl)-N-[4-(2-{[2-methoxy-4-(2-oxo-1,3-oxazolidin-3-yl)phenyl]-amino}[1,2,4]triazolo[1,5-a]pyridin-6-yl)phenyl]propanamide,
(−)-2-(4-fluorophenyl)-3-hydroxy-N-[4-(2-{[4-(methylsulfonyl)-2-(2,2,2-trifluoroethoxy)phenyl]amino}[1,2,4]triazolo[1,5-a]pyridin-6-yl)phenyl]propanamide,
(2R)-2-amino-2-(4-fluorophenyl)-N-[4-(2-{[2-methoxy-4-(methylsulfonyl)phenyl]amino}[1,2,4]triazolo[1,5-a]pyridin-6-yl)phenyl]ethanamide,
4-{[6-(4-{[(2R)-2-(4-fluorophenyl)propanoyl]amino}phenyl)[1,2,4]triazolo[1,5-a]pyridin-2-yl]amino}-3-methoxy-N,N-dimethylbenzamide,
(2R)-2-(4-fluorophenyl)-N-[4-(2-{[2-methoxy-4-(pyrrolidin-1-ylcarbonyl)phenyl]amino}[1,2,4]triazolo[1,5-a]pyridin-6-yl)phenyl]propanamide,
(2R)-N-{4-[2-({4-[(3-fluoroazetidin-1-yl)carbonyl]-2-(2,2,2-trifluoroethoxy)phenyl}amino)[1,2,4]triazolo[1,5-a]pyridin-6-yl]phenyl}-2-(4-fluorophenyl)propanamide,
(2R)-2-(4-fluorophenyl)-N-{4-[2-({4-[(3-hydroxyazetidin-1-yl)carbonyl]-2-(2,2,2-trifluoroethoxy)phenyl}amino)[1,2,4]triazolo[1,5-a]pyridin-6-yl]phenyl}propanamide,
(2R)-2-(4-fluorophenyl)-N-[4-(2-{[4-(pyrrolidin-1-ylcarbonyl)-2-(2,2,2-trifluoroethoxy)phenyl]amino}[1,2,4]triazolo[1,5-a]pyridin-6-yl)phenyl]propanamide,
(2S)-2-(4-fluorophenyl)-3-hydroxy-N-[4-(2-{[2-methoxy-4-(methylsulfonyl)phenyl]amino}[1,2,4]triazolo[1,5-a]pyridin-6-yl)phenyl]propanamide,
(2S)-N-{4-[2-({4-[(3-fluoroazetidin-1 -yl)carbonyl]-2-(2,2,2-trifluoroethoxy)phenyl}amino)[1,2,4]triazolo[1,5 -a]pyridin-6-yl]phenyl}-2-(4-fluorophenyl)-3- hydroxypropanamide,
(2R)-2-amino-2-(4-fluorophenyl)-N-[4-(2-{[4-(methylsulfonyl)-2-(2,2,2-trifluoroethoxy)phenyl]amino}[1,2,4]triazolo[1,5-a]pyridin-6-yl)phenyl]ethanamide,
(2R)-2-amino-2-(4-fluorophenyl)-N-[4-(2-{[2-methoxy-4-(2-oxo-1,3-oxazolidin-3-yl)phenyl]amino}[1,2,4]triazolo[1,5-a]pyridin-6-yl)phenyl]ethanamide,
(2R)-2-amino-N-{4-[2-({4-[(3-fluoroazetidin-1 -yl)carbonyl]-2-methoxyphenyl}amino)[1,2,4]triazolo[1,5-a]pyridin-6-yl]phenyl}-2-(4-fluorophenyl)ethanamide,
(2R)-2-amino-N-[4-(2-{[4-(azetidin-1 -ylcarbonyl)-2-methoxyphenyl]amino}[1,2,4]triazolo[1,5-a]pyridin-6-yl)phenyl]-2-(4-fluorophenyl)ethanamide,
(2R)-2-amino-2-(4-fluorophenyl)-N-[4-(2-{[2-methoxy-4-(pyrrolidin-1-ylcarbonyl)phenyl]amino}[1,2,4]triazolo[1,5-a]pyridin-6-yl)phenyl]ethanamide,
(2R)-2-amino-N-{4-[2-({4-[(3-fluoroazetidin-1-yl)carbonyl]-2-(2,2,2-trifluoroethoxy)phenyl}amino)[1,2,4]triazolo[1,5 -a]pyridin-6-yl]phenyl}-2-(4-fluorophenyl)ethanamide, and
(2R)-2-amino-2-(4-fluorophenyl)-N- [4-(2-{[4-(pyrrolidin-1-ylcarbonyl)-2-(2,2,2-trifluoroethoxy)phenyl]amino}[1,2,4]triazolo[1,5-a]pyridin-6-yl)phenyl]ethanamide,
or an N-oxide, a hydrate, a solvate, or a salt thereof, or a mixture of same.

7. The combination according to claim 1, wherein compound A is (2R)-2-(4-fluorophenyl)-N-[4-(2-{[2-methoxy-4-(methylsulfonyl)phenyl]amino}[1,2,4]triazolo[1,5-a]pyridin-6-yl)phenyl]propanamide or an N-oxide, a hydrate, a solvate, or a salt thereof, or a mixture of same.

8. The combination according to claim 1, further comprising cisplatin.

9. (canceled)

10. (canceled)

11. A method of treatment of pancreatic cancer, glioblastoma, ovarian cancer, non-small cell lung carcinoma, breast cancer or gastric cancer in a subject in need thereof, comprising administering to said subject a therapeutically effective amount of a combination according to claim 1.

12. A kit comprising a combination of:
component A: one or more compounds A, as defined in claim 1;
and
component B: one or more mitotic inhibitors;
and, optionally, one or more further pharmaceutical agents C;
in which optionally all or either of said components A and B are in the form of a pharmaceutical formulation which is ready for use to be administered simultaneously, concurrently, separately or sequentially.

13. The kit according to claim 12, wherein the mitotic inhibitor is selected from docetaxel and paclitaxel, and the optional pharmaceutical agent C is cisplatin.

14. The combination according to claim 2, wherein the vinca alkaloid is selected from vinblastine, vincristine, vindesine, vinorelbine, desoxyvincaminol, vincaminol, vinburnine, vincamajine, vineridine, and vinburnine.

15. The combination according to claim 3, wherein the taxane is selected from docetaxel, paclitaxel, and their analogues.
